(19) United States
(12) Patent Application Publication
(10) Pub. No.: US 2002/0120232 A1
(43) Pub. Date: Aug. 29, 2002

(54) Cannula, in particular for use as a retrograde cardioplegia cannula

(76) Inventors: Gerd Stumpp, Hirrlingen (DE); Dietmar Klietsch, Herrenberg (DE)

Correspondence Address: STRIKER, STRIKER & STENBY, 103 East Neck Road, Huntington, NY, 11743, US

(21) Appl. No.: 10/007,213

(22) Filed: Nov. 8, 2001

(30) Foreign Application Priority Data
Nov. 8, 2000 (EP) 00 124401.1

(51) Int. Cl. A61M 37/00

(52) U.S. Cl. 604/95.03

(57) ABSTRACT

A cannula (10), in particular for use a retrograde cardioplegia cannula, having a balloon (12), disposed in the region of its tip (11) and inflatable by means of a fluid fed in through the cannula (10), for sealing off the space between the cannula (10) and a vessel and for positioning the cannula (10) in the vessel, wherein the balloon (12) is provided with a plurality of openings (19), through which the fluid fed in by means of the cannula (10) can emerge into the vessel.

The invention relates to a cannula, in particular for use a retrograde cardioplegia cannula, having a balloon, disposed in the region of its tip and inflatable by means of a fluid fed in through the cannula, for sealing off the space between the cannula and a vessel and for positioning the cannula in the vessel.

Such cannulas are already known in cardiac surgery. They are used as retrograde cardioplegia cannulas, with the aid of which cardioplegia fluid for cooling the heart muscle and for supplying the heart with minerals is introduced into the body of the patient. To that end, the cannulas have an outlet opening at the tip for the cardioplegia fluid. They are also provided with further radial openings in the region of the balloon, through which cardioplegia fluid can flow into the balloon in order to inflate it and to achieve both the desired sealing off of the vessel and the positioning of the cannula. However, the known cannulas have the disadvantage that the air that is always present in the balloon before it is filled can escape only extremely poorly, causing delays in inflating the balloon.

The object of the present invention is to improve a cannula of the type described at the outset in such a way that rapid filling and inflation of the balloon is possible.

This object is attained according to the invention, by means of a cannula of the type defined at the outset, in that the balloon is provided with a plurality of openings, through which the fluid fed in by means of the cannula can emerge into the vessel.

The holes present in the balloon enable the air present to escape rapidly and completely as soon as the fluid flows into the balloon. At the same time, these openings also serve as outlet openings for the cardioplegia fluid. Thus rapid filling of the balloon and hence rapid sealing off of the vessel and secure positioning of the cannula in the vessel are assured.

Preferably, the openings can be disposed in the front region of the balloon, distributed uniformly over the circumference thereof. The result is an extremely uniform distribution of the cardioplegia fluid.

Further advantages are attained if the openings in the balloon are the sole outlet openings for the fluid, that is, if there are no further openings in the region of the cannula tip for the fluid. Because the fluid is dammed from the tip of the cannula back into the balloon, the filling of the balloon is speeded up still further.

For gentle placement of the cannula, it is also expedient if the tip of the cannula is rounded.

A preferred exemplary embodiment of a cannula of the invention will now be described in further detail in conjunction with the drawing.

Shown are:

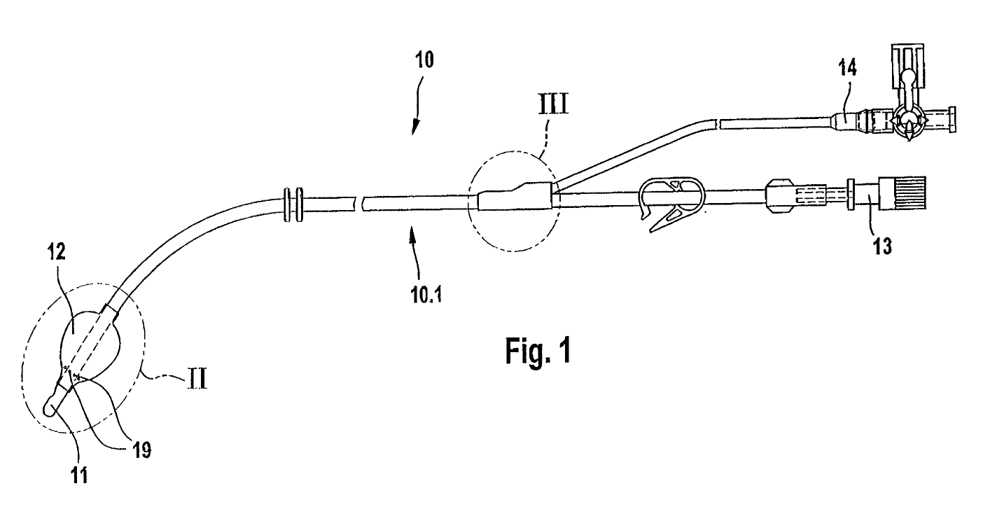

FIG. 1, a side view of a cannula of the invention;

FIG. 2, an enlarged sectional view of the cannula of FIG. 1 in the region marked II;

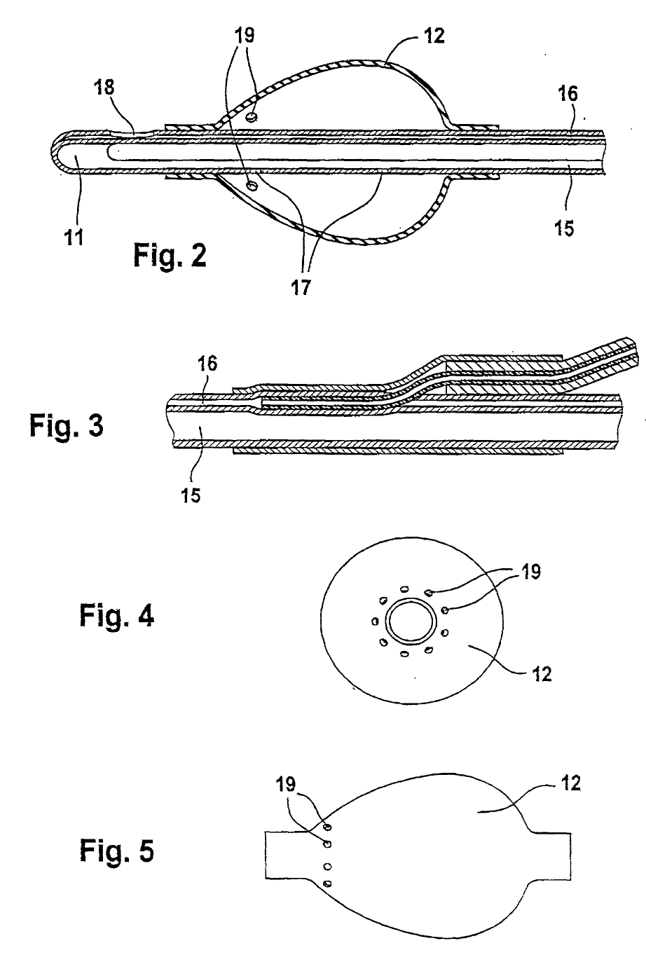

FIG. 3, an enlarged sectional view of the cannula of FIG. 1 in the region marked III;

FIG. 4, a front view of the balloon of the cannula of FIG. 1;

FIG. 5, a side view of the balloon of FIG. 4.

The cannula 10 of FIG. 1 has a balloon 12 in the region of its tip 11; with the aid of the balloon, the cannula 10 can be sealed off from a vessel and positioned. On the opposite end, the cannula 10 has a connection for feeding in a fluid, such as a cardioplegia fluid. A further connection 14 is also provided for pressure measurement; it discharges into the cannula 10 itself in the region marked III.

As FIGS. 2 and 3 show, the cannula 10 is divided longitudinally into two separate hollow chambers 15 and 16; the larger hollow chamber 15 is intended for guiding the fluid from the connection point 13, and the smaller hollow chamber 16 is intended for measuring pressure. As FIG. 2 shows, the cannula tube 10.1 has radial openings 17 in the region of the balloon 12; through these openings, the fluid can enter the balloon 12 from the hollow chamber 15. The fluid for pressure measurement enters through an opening 18 in the region of the tip 11 of the cannula 10.

From FIGS. 1, 4 and 5, it can be seen that the balloon 12 is provided in its front region with radial openings 19, distributed over its circumference, through which in the filling of the balloon 12, first the air present there and

1. A cannula, in particular for use a retrograde cardioplegia cannula, having a balloon (12), disposed in the region of its tip (11) and inflatable by means of a fluid fed in through the cannula (10), for sealing off the space between the cannula (10) and a vessel and for positioning the cannula (10) in the vessel, characterized in that the balloon (12) is provided with a plurality of openings (19), through which the fluid fed in by means of the cannula (10) can emerge into the vessel.

2. The cannula of claim 1, characterized in that the openings (19) are disposed in the front region of the balloon (12), distributed uniformly over the circumference thereof.

3. The cannula of claim 1, characterized in that the openings (19) in the balloon (12) are the sole outlet openings for the fluid.

4. The cannula of claim 1, characterized in that the tip (11) of the cannula is rounded.
